(19) United States
(12) Patent Application Publication
(10) Pub. No.: US 2025/0099171 A1
(43) Pub. Date: Mar. 27, 2025

(54) MAPPING AND ABLATION ELECTROPORATION CATHETER

(71) Applicant: Boston Scientific Scimed, Inc., Maple Grove, MN (US)

(72) Inventor: Gary L. Long, Cincinnati, OH (US)

(21) Appl. No.: 18/892,116

(22) Filed: Sep. 20, 2024

Related U.S. Application Data
(60) Provisional application No. 63/584,444, filed on Sep. 21, 2023.

(51) Int. Cl. A61B 18/14; A61B 18/00

(52) U.S. Cl. CPC A61B 18/1492; A61B 2018/0022; A61B 2018/00267; A61B 2018/00375; A61B 2018/00577; A61B 2018/00613; A61B 2018/00642; A61B 2018/00839; A61B 2018/1467

(57) ABSTRACT

An electroporation catheter for facilitating ablation of cardiac tissue is disclosed. The electroporation catheter includes an elongated shaft having a distal region. An inflatable member and electrode assembly are coupled to the distal region. The inflatable member is transitionable between an inflated configuration and an uninflated configuration. The electrode assembly includes electrodes and flexible support members. The electrodes include sensing electrodes disposed on the flexible support members and ablation electrodes configured to generate an electric field. The flexible support members are transitionable between an expanded configuration and a collapsed configuration. The flexible support members form a cavity, and the inflatable member is disposed within the cavity.

### CROSS REFERENCE TO RELATED APPLICATIONS

The present application claims priority to U.S. Provisional Patent Application No. 63/584,444, filed Sep. 21, 2023, the entire disclosure of which is incorporated herein by reference.

### TECHNICAL FIELD

The present disclosure relates to medical systems and methods for facilitating ablation of tissue in a patient. More specifically, the present disclosure relates to medical systems, catheters, and methods for facilitating ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. For instance, catheter ablation is a minimally invasive electrophysiological procedure that can be used to treat a variety of heart conditions such as atrial fibrillation via pulmonary vein isolation. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques can indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells in order to increase the permeability of the cell membrane. The electroporation can be reversible or irreversible, depending on the strength and duration of the electric field. If the electroporation is reversible, the temporarily increased permeability of the cell membrane can be used to introduce chemicals, drugs, or deoxyribonucleic acid (DNA) into the cell, prior to the cell healing and recovering. Tissue recovery can occur over minutes, hours, or days after the ablation is completed. If the electroporation is irreversible, the affected cells are killed, such as via form of cell death, such as perhaps programmed cell death through apoptosis for example, or such as traumatic cell death through necrosis for example.

Irreversible electroporation can be used as a nonthermal ablation technique. In irreversible electroporation, trains of short, high voltage pulses are used to generate electric fields that are strong enough to kill cells. In ablation of cardiac tissue, irreversible electroporation can be a relatively safe and effective alternative to the indiscriminate killing of thermal ablation techniques, such as RF ablation and cryoablation. Irreversible electroporation can be used to kill targeted tissue, such as myocardium tissue, by using a selected electric field strength and duration that is effective to kill the targeted tissue but is not effective to permanently damage other cells or tissue, such as non-targeted myocardium tissue, red blood cells, vascular smooth muscle tissue, endothelium tissue, and nerve cells.

### SUMMARY

In Example 1, an electroporation catheter for ablation of cardiac tissue. The electroporation catheter comprising: an elongated shaft having a distal region; a sensing electrode assembly extending distally from the distal region of the elongated shaft and defining an interior region, the sensing electrode assembly defining a distally located central hub portion and a plurality of flexible support members each including a distal end portion extending from the central hub portion, and a proximal end portion attached to and constrained by the shaft, each of the plurality of flexible support members including a plurality of sensing electrodes; wherein the flexible support members are transitionable between an expanded configuration and a collapsed configuration, the plurality of flexible support members forming the interior region in the expanded configuration; an inflatable member having a proximal portion and a distal portion, the inflatable member disposed within the interior region and operably coupled to the distal region, the inflatable member transitionable between an inflated configuration and an uninflated configuration; and an ablation electrode assembly operably coupled to the inflatable member, the ablation electrode assembly having a proximal ablation electrode disposed proximal to the inflatable member and a distal ablation electrode disposed distal to the inflatable member, the ablation electrodes configured to generate an electric field to accomplish pulsed field ablation.

In Example 2, the electroporation catheter of Example 1, wherein the flexible support members in the expanded configuration are configured to engage the walls of a pulmonary vein ostium.

In Example 3, the electroporation catheter of any of Examples 1-2, wherein the inflatable member in the inflated configuration is configured to engage the wall of a pulmonary vein ostium.

In Example 4, the electroporation catheter of any of Examples 1-3, wherein the inflatable member in the inflated configuration is configured to substantially occlude the pulmonary vein ostium.

In Example 5, the electroporation catheter of any of Examples 1-4, wherein the plurality of flexible support members form a basket having the cavity within the basket, and the inflatable member disposed within the basket.

In Example 6, the electroporation catheter of any of Examples 1-5, wherein the plurality of flexible support members includes a plurality of splines.

In Example 7, the electroporation catheter of any of Examples 1-6, wherein each spline includes a set of the plurality of sensing electrodes.

In Example 8, the electroporation catheter of any of Examples 1-7, wherein the plurality of ablation electrodes includes one distal ablation electrode disposed distal to the inflatable member and one proximal ablation electrode disposed proximal to the inflatable member.

In Example 9, the electroporation catheter of any of Examples 5-8, wherein the distal ablation electrode is disposed on the central hub portion.

In Example 10, the electroporation catheter of any of Examples 5-9, wherein the proximal ablation electrode is coupled to the shaft proximal to the flexible support members.

In Example 11, the electroporation catheter of any of Examples 1-10, wherein the inflatable member is formed of an insulating material.

In Example 12, the electroporation catheter of Example 11, wherein the inflatable member in the inflated configuration includes a non-conductive fluid.

In Example 13, the electroporation catheter of any of Examples 1-12, wherein the electroporation catheter is included in an electrophysiology system including an electroporation console configured to generate pulsed electrical signals operably coupled to the plurality of ablation electrodes for electroporation ablation and an electroanatomical mapping system operably coupled to the plurality of mapping electrodes and configured to receive cardiac signals.

In Example 14, the electroporation catheter of Example 13, wherein the electrophysiological system further comprises an infusion device, wherein the inflatable member is fluidically coupled to the infusion device to transition between the inflated configuration and the uninflated configuration in response to an infusion of fluid from the infusion device.

In Example 15, the electroporation catheter of any of Examples 5-14, wherein the inflatable member in the inflated configuration is configured to substantially occlude the walls of a pulmonary vein ostium while the plurality of ablation electrodes deliver ablation energy to the walls of the pulmonary vein ostium and while the plurality of sensing electrodes obtain cardiac signals.

In Example 16, an electroporation catheter for ablation of cardiac tissue. The electroporation catheter comprising: an elongated shaft having a distal region; a sensing electrode assembly extending distally from the distal region of the elongated shaft and defining an interior region, the sensing electrode assembly defining a distally located central hub portion and a plurality of flexible support members each including a distal end portion extending from the central hub portion, and a proximal end portion attached to and constrained by the shaft, each of the plurality of flexible support members including a plurality of sensing electrodes; wherein the flexible support members are transitionable between an expanded configuration and a collapsed configuration, the plurality of flexible support members forming the interior region in the expanded configuration; an inflatable member having a proximal portion and a distal portion, the inflatable member disposed within the interior region and operably coupled to the distal region, the inflatable member transitionable between an inflated configuration and an uninflated configuration; and an ablation electrode assembly operably coupled to the inflatable member, the ablation electrode assembly having a proximal ablation electrode disposed proximal to the inflatable member and a distal ablation electrode disposed distal to the inflatable member, the ablation electrodes configured to generate an electric field to accomplish pulsed field ablation.

In Example 17, the electroporation catheter of Example 16, wherein the plurality of flexible support members form a basket having the cavity within the basket, and the inflatable member disposed within the basket.

In Example 18, the electroporation catheter of Example 17, wherein the basket includes a distal tip, and the first ablation electrode is disposed on the distal tip of the basket.

In Example 19, the electroporation catheter of Example 18, wherein the basket is operably coupled to the shaft, and the proximal ablation electrode is coupled to the shaft proximal to the basket.

In Example 20, the electroporation catheter of Example 16, wherein the distal ablation electrode is configured as one of a cathode and an anode and wherein the proximal ablation electrode is configured as the other of the cathode and the anode, and wherein the electroporation catheter is operable in a bipolar mode.

In Example 21, the electroporation catheter of Example 16, wherein the plurality of ablation electrodes includes one distal ablation electrode disposed distal to the inflatable member and one proximal ablation electrode disposed proximal to the inflatable member.

In Example 22, the electroporation catheter of Example 16, wherein the inflatable member is formed of an insulating material.

In Example 23, the electroporation catheter of Example 22, wherein the inflatable member in the inflated configuration includes a non-conductive fluid.

In Example 24, the electroporation catheter of Example 16, wherein the plurality of flexible support members includes a plurality of splines.

In Example 25, the electroporation catheter of Example 24, wherein each spline includes a set of the plurality of sensing electrodes longitudinally spaced along the spline.

In Example 26, an electrophysiology system, comprising: an electroporation console configured to generate pulsed electrical signals for electroporation ablation; and an electroporation catheter operably coupled to the electroporation console and the electroanatomical mapping system, the electroporation catheter comprising: an elongated shaft having a distal region; a sensing electrode assembly extending distally from the distal region of the elongated shaft and defining an interior region, the sensing electrode assembly defining a distally located central hub portion and a plurality of flexible support members each including a distal end portion extending from the central hub portion, and a proximal end portion attached to and constrained by the shaft, each of the plurality of flexible support members including a plurality of sensing electrodes; wherein the flexible support members are transitionable between an expanded configuration and a collapsed configuration, the plurality of flexible support members forming the interior region in the expanded configuration; an inflatable member having a proximal portion and a distal portion, the inflatable member disposed within the interior region and operably coupled to the distal region, the inflatable member transitionable between an inflated configuration and an uninflated configuration; and an ablation electrode assembly operably coupled to the inflatable member, the ablation electrode assembly having a proximal ablation electrode disposed proximal to the inflatable member and a distal ablation electrode disposed distal to the inflatable member, the ablation electrodes configured to generate an electric field to accomplish pulsed field ablation.

In Example 27, the electrophysiological system of Example 26, and further comprising an infusion device, wherein the inflatable member is fluidically coupled to the infusion device to transition between the inflated configuration and the uninflated configuration in response to an infusion of fluid from the infusion device.

In Example 28, the electrophysiological system of Example 26, wherein an ablation electrode in the plurality of ablation electrodes is configured as a cathode and another ablation electrode in the plurality of ablation electrodes is configured as an anode, and the electroporation catheter is operable in a bipolar mode.

In Example 29, a method for an electrophysiological procedure, the method comprising: providing an electroporation catheter comprising: an elongated shaft having a distal region; a sensing electrode assembly extending distally from the distal region of the elongated shaft and defining an interior region, the sensing electrode assembly defining a distally located central hub portion and a plurality of flexible support members each including a distal end portion extending from the central hub portion, and a proximal end portion attached to and constrained by the shaft, each of the plurality of flexible support members including a plurality of sensing electrodes; wherein the flexible support members are transitionable between an expanded configuration and a collapsed configuration, the plurality of flexible support members forming the interior region in the expanded configuration; an inflatable member having a proximal portion and a distal portion, the inflatable member disposed within the interior region and operably coupled to the distal region, the inflatable member transitionable between an inflated configuration and an uninflated configuration; and an ablation electrode assembly operably coupled to the inflatable member, the ablation electrode assembly having a proximal ablation electrode disposed proximal to the inflatable member and a distal ablation electrode disposed distal to the inflatable member, the ablation electrodes configured to generate an electric field to accomplish pulsed field ablation; deploying the electroporation catheter in the expanded configuration and the inflatable member in the inflated configuration into a pulmonary vein ostium; delivering electroporation ablation energy to the ablation electrodes to ablate a wall of the pulmonary vein ostium with the deployed electroporation catheter in the expanded configuration and the inflatable member in the inflated configuration; and obtaining cardiac signals with the sensing electrodes with the deployed electroporation catheter in the expanded configuration.

In Example 30, the method of Example 29, wherein deploying the electroporation catheter includes inflating the inflatable member into the cavity after expanding the flexible support members to form the cavity.

In Example 31, the method of Example 29, wherein deploying the electroporation catheter includes infusing the inflatable member with a fluid.

In Example 32, the method of Example 29, wherein the flexible support members include splines and deploying the electroporation catheter includes forming the basket in the expanded state.

In Example 33, the method of Example 29, wherein delivering electroporation ablation energy to the ablation electrodes to ablate a wall of the pulmonary vein ostium with the deployed electroporation catheter in the expanded configuration and the inflatable member in the inflated configuration includes occluding the pulmonary vein ostium.

In Example 34, the method of Example 29, wherein the inflatable member is formed of an insulating material, and wherein delivering electroporation ablation energy to the ablation electrodes to ablate a wall of the pulmonary vein ostium includes directing the electric field toward the wall of the pulmonary vein ostium with the inflatable member.

In Example 35, the method of Example 29, wherein the obtaining cardiac signals with the sensing electrodes includes occluding the pulmonary vein ostium.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system, in accordance with embodiments of the subject matter of the disclosure.

FIG. 2 is a schematic diagram illustrating an example electroporation catheter that can be used with the example electrophysiology system of FIG. 1.

FIG. 3 is a schematic diagram illustrating another example electroporation catheter that can be used with the example electrophysiology system of FIG. 1.

FIG. 4 is a schematic diagram illustrating still another example electroporation catheter that can be used with the example electrophysiology system of FIG. 1.

FIG. 5 is a schematic diagram illustrating the example electroporation catheter of FIG. 2 disposed in a pulmonary vein ostium.

FIG. 6 is a schematic diagram illustrating still another example electroporation catheter that can be used with the example electrophysiology system of FIG. 1.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are considered to be within the ambit of the present disclosure.

As applied to electrophysiology systems, irreversible electroporation uses high voltage, short pulses to kill cells such as myocardium through apoptosis while sparing other adjacent tissues including the esophageal vascular smooth muscle and endothelium. Irreversible electroporation treatment can be delivered in multiple therapy sections. A therapy section, which may have a duration on the order of milliseconds, may include a plurality of electrical pulses, such as a few dozen pulses, generated and delivered by an electroporation device, which is powered by an electroporation generator, to generate an electric field of sufficient strength to create transmural lesions. In one example, one or more catheters may be advanced in a minimally invasive fashion through vasculature to a target location, such as in the heart. The methods described here may include introducing a device into an endocardial space of the heart and disposing the device at the ostium of a pulmonary vein. A pulse waveform may be generated and delivered to electrodes of the device to ablate tissue. The electrodes can be configured in anode-cathode subsets. In some examples, the pulse waveform may be generated in synchronization with a pacing signal of the heart to avoid disruption of the sinus rhythm of the heart.

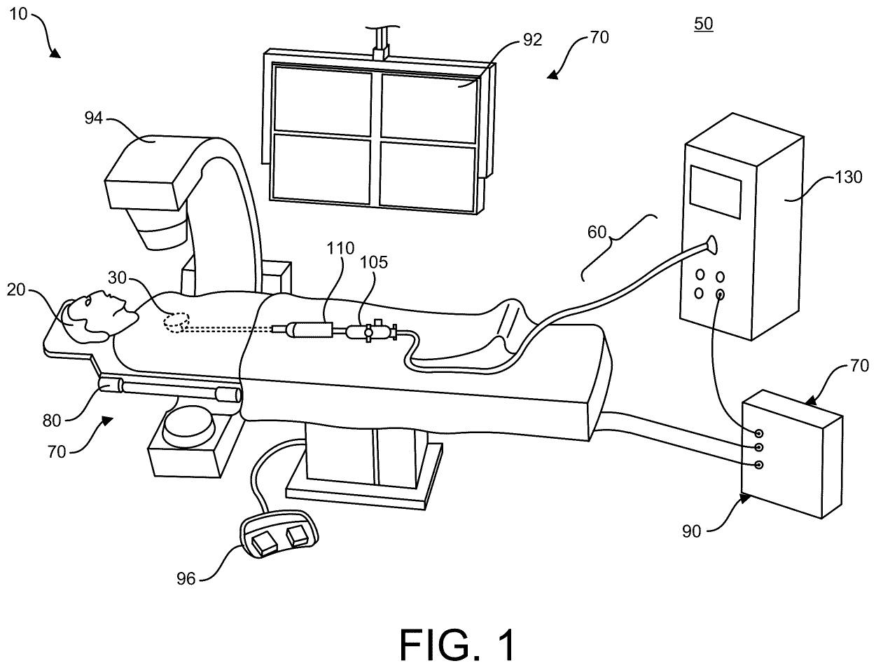

FIG. 1 illustrates an example clinical setting 10 for treating a patient 20, such as for treating a heart 30 of the patient 20, using an electrophysiology system 50, in accordance with the disclosure. The electrophysiology system 50 includes an electroporation catheter system 60 and an electro-anatomical mapping (EAM) system 70. The example electroporation catheter system 60 includes an electroporation catheter 105, an introducer sheath 110, and an electroporation console 130. Additionally, the electroporation catheter system 60 includes various connecting elements, such as cables and tubing, that operably connect the components of the electroporation catheter system 60 to one another and to the components of the EAM system 70. In general, the EAM mapping system 70 includes a localization field generator 80, a mapping and navigation controller 90, and a display 92. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1.

The electroporation catheter system 60 is configured to deliver ablation electric field energy to targeted tissue in the patient's heart 30 to create cell death in tissue, for example, rendering the tissue incapable of conducting electrical signals. Also, the electroporation catheter system 60 is configured to generate electric fields using the electroporation catheter 105 to create and present, on the display 92, an electro-anatomical map of the patient's heart to aid a clinician in planning ablation by irreversible electroporation using the electroporation catheter 105 prior to delivering ablation electric field energy. In embodiments, the electroporation catheter system 60 is configured to generate the electric fields based on characteristics of the electroporation catheter 105 and the position of the electroporation catheter 105 in the patient 20, such as in the heart 30 of the patient 20. The electroporation catheter system 60 is configured to generate graphical representations of the electroporation catheter and the electro-anatomical map based on characteristics of the electroporation catheter 105 and the position of the electroporation catheter 105 in the patient 20, such as in the heart 30 of the patient 20, and the characteristics of the tissue surrounding the catheter 105, such as measured impedances of the tissue. The electroporation catheter system 60 can include additional features. For instance, the electroporation console 130 can include, or be operably coupled to, an infusion device that may be fluidically coupled to catheters having inflatable members, such as balloons, via tubing. The infusion device can selectively inflate or deflate the inflatable member via a pump or other mechanism to provide a fluid from a reservoir to the inflatable member. In one example, the electroporation catheter 105 is a mapping and ablation catheter, which can be deployed in mapping procedures in cooperation with the EAM system 70 as well as to deliver ablation electric field energy and ablate tissue via irreversible electroporation.

The introducer sheath 110 is operable to provide a delivery conduit through which the electroporation catheter 105 can be deployed to the specific target sites within the patient's heart 30. Access to the patient's heart can be obtained through a vessel, such as a peripheral artery or vein often in the groin or possibly in the shoulder or neck. Once access to the vessel is obtained, the electroporation catheter 105 can be navigated to within the patient's heart, such as within a chamber of the heart. In pulmonary vein isolation, the catheter can be maneuvered from the right atrium to the left atrium where the pulmonary veins typically connect to the heart. For instance, the left atrium includes ostia, or openings, and each ostium corresponds with a pulmonary vein. Oxygen-rich blood travels through the ostia into the left atrium. Atrial fibrillation is an abnormal heart rhythm that begins in the pulmonary veins or in the region of the ostium. The electroporation catheter can be maneuvered to a pulmonary vein ostium to treat atrial fibrillation.

The example electroporation catheter 105 includes an elongated catheter shaft and distal end region configured to be deployed proximate target tissue, such as within a chamber of the patient's heart or the wall of a pulmonary vein ostium. The shaft can extend from an access point in the patient to the target tissue and generally defines a longitudinal axis of the electroporation catheter 105. A proximal end region of the catheter can include a handle having user manipulatable controls for the catheter 105. The distal end region may include a basket, balloon, spline, configured tip, or other electrode deployment mechanism coupled to the shaft. The electrode deployment mechanism includes an electrode assembly, or array, comprising an electrode. For example, the electrode assembly can include a plurality of spaced-apart electrodes or multiple spaced-apart sets or groups of spaced-apart electrodes. In some examples, an electrode, such as a plurality of spaced-apart electrodes, can be deployed on the catheter shaft in addition to or instead of an electrode on the electrode deployment mechanism. For instance, the electrode deployment mechanism includes a plurality of flexible support members configured to form a basket, and at least a some of the electrodes are disposed on the flexible support members.

The electroporation catheter 105 is configurable in a plurality of configurations. For example, when the distal end region of the catheter 105 is within a sheath as a catheter assembly, such as to travel to the patient to the chamber of the heart, the electrode deployment mechanism and electrode assembly are in a collapsed configuration to fit within the sheath. Once the catheter has reached the destination in the chamber of the heart, for example, the sheath is retracted from the distal region of the catheter 105 (or the shaft catheter is extended past the sheath) and the electrode deployment mechanism and electrode assembly can be arranged in an expanded configuration. The electrode assembly has a collapsed shape when the catheter 105 is in the collapsed state and an expanded shape when the catheter 105 is in the expanded state. In some examples, the electrode assembly has more than two states.

In one example, the plurality of electrodes can be formed of a conductive, solid-surface, biocompatible material and are spaced-apart across insulators. Each of the plurality of electrodes is electrically coupled to a corresponding elongated lead conductor that extend along the shaft to a catheter proximal end. In one example, each electrode of the spaced-apart electrodes corresponds with a separate, single lead conductor. In another example, a plurality of electrodes may be coupled to a single lead conductor. Other configurations are contemplated. The plurality of lead conductors can be insulated from one another within an insulating sheath along the catheter shaft, such as with an insulating polymer sheath. The lead conductors can be electrically coupled to plug in the proximal region of the electroporation catheter 105, such as a plug configured to be mechanically and electrically coupled to the electroporation console 130, for example, either directly or via intermediary electrical conductors such as cabling.

The electroporation console 130 includes a controller, such one or more controllers, processors, or computers, that executes instructions or code, such as processor-executable instructions, out of a non-transitory computer readable medium, such as a memory device, or memory, to cause, such as control or perform, the aspects of the electroporation catheter system 60. In one example, the electroporation console 130 is configured to provide an electrical signal, such as a plurality of concurrent or space-apart-time electrical signals, to the electrically connected electroporation catheter 105 along lead conductors to the spaced-apart electrodes. The spaced-apart electrodes are configured to generate a selected electrical field proximate the target tissue, based on the electrical signals from the electroporation console 130, such as to effect ablation.

The electroporation console 130 can generate electrical signals and select which electrodes in the electrode array will receive the electrical signals. A first electrode, or first group of electrodes, can be selected to be an anode and a different, second electrode, or second group of electrodes, can be selected to be a cathode, such that electrical fields can be generated between the anode and cathode based on signals, such as pulses, provided to the electrodes from the electroporation console 130. The console 130 provides electric pulses of different lengths and magnitudes to the electrodes on the catheter 105. The electric pulses can be provided in a continuous stream of pulses or in multiple, separate trains of pulses. Pulse parameters of interest include the number of pulses, the duty cycle of the pulses, the spacing of pulse trains, the voltage or magnitude of the pulses including the peak voltages, and the duration of the voltages. For example, the console 130 can select two or more electrodes of the electrode assembly and provides pulses to the selected electrodes to generate electric fields between the selected electrodes.

In an ablation mode, the console can select electrodes to provide pulsed field ablation (PFA). For example, PFA can be performed with monophasic waveforms and biphasic waveforms. Without being bound to a particular theory, electric field strengths in the range of generally 200-250 volts per centimeter (V/cm) with microsecond-scale pulse duration have been demonstrated to provide reversible electroporation in cardiac tissue. Electric field strengths at approximately 400 V/cm have been demonstrated to provide irreversible electroporation in cardiac tissue of interest, such as targeted myocardium tissue and endocardium tissue, with demonstrable sparing of red blood cells, vascular smooth muscle tissue, endothelium tissue, nerves and other non-targeted proximate tissue.

Additionally, the electrode assembly on catheter 105 can be operated in a selected mode such as monopolar mode or bipolar mode. During monopolar operation of the catheter 105, an electrode, a group of electrodes, or the entire electrode assembly are configured as one of an anode or a cathode. None of the electrodes in the electrode assembly are configured as a the other of the cathode or the anode. Instead, the other of the cathode or the anode is provided in the form of a pad dispersive electrode located on the patient, typically on the back, buttocks, or other suitable anatomical location during electroporation. An electrical field is formed between an activated electrode of the electrode assembly and the pad dispersive electrode. In an alternative configuration, a return electrode, such as a plurality of return electrodes, can be disposed on the shaft of the catheter. An electrical field is formed between an activated electrode of the electrode assembly and the return electrode on the shaft of the catheter 105. During bipolar operation of the catheter 105, a first set of one or more electrodes of the electrode assembly, is configured as the anode and a second set of one or more electrodes of the electrode assembly, is configured as the cathode, to generate the electric field. In this example, a pad dispersive electrode is not used, and the electrical field is not extended in the patient's body, but rather through a localized portion of tissue proximate the electrode assembly.

In the illustrated examples, the catheter 105 is a mapping and ablation catheter, and the electrodes can include ablation electrodes that are configured to deliver ablation electric field energy and mapping electrodes for mapping purposes. In some configurations, the mapping electrodes are configured to be used to collect electrical signals to be used to generate via the operably coupled EAM system 70, and display via the operably coupled display 92, detailed three-dimensional geometric anatomical maps or representations of the cardiac chambers as well as electro-anatomical maps in which cardiac electrical activity of interest is superimposed on the geometric anatomical maps. In some examples, an electrode can operate as an ablation electrode in an ablation mode of the electrophysiology system 50 and as a mapping electrode in a mapping mode of the system 50. Mapping electrodes on the electroporation catheter 105 can measure electrical signals and generate output signals that can be processed by the mapping and navigation controller 90 to generate an electro-anatomical map. In some instances, electro-anatomical maps are generated before ablation for determining the electrical activity of the cardiac tissue within a chamber of interest. In some instances, electro-anatomical maps are generated after ablation in verifying the desired change in electrical activity of the ablated tissue and the chamber. The mapping electrodes may also be used to determine the position of the catheter 105 in three-dimensional space within the body. For example, when the operator moves the distal end of the catheter 105 within a cardiac chamber of interest, the boundaries of catheter movement can be used by the mapping and navigation controller 90 to form the anatomical map of the chamber. The chamber anatomical map may be used to facilitate navigation of the catheter 105 without the use of ionizing radiation such as with fluoroscopy, and for tagging locations of ablations as they are completed in order to guide spacing of ablations and aid the clinician in ablating the anatomy of interest. In some examples, an electrode in the electrode assembly can be configured to only perform an ablation or the electrode in the electrode assembly can be configured to only perform mapping.

The EAM system 70 is configured to generate the electro-anatomical map for display on the display 92. The EAM system 70 is operable to track the location of the various components of the electroporation catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative example, the EAM system 70 can include the RHYTHMIA™ HDx mapping system marketed by Boston Scientific Corporation. Also, the mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 generates a localization field, via the field generator 80, to define a localization volume about the heart 30, and a location sensor or sensing element on a tracked device, such as sensors on the electroporation catheter 105, generate an output that can be processed by the mapping and navigation controller 90 to track the location of the sensor, and consequently, the corresponding device, within the localization volume. In the illustrated example, the device tracking is accomplished using magnetic tracking techniques, in which the field generator 80 is a magnetic field generator that generates a magnetic field defining the localization volume, and location sensors on the tracked devices are magnetic field sensors.

In other examples, impedance tracking methodologies may be employed to track the locations of the various devices. In such examples, the localization field is an electric field generated, for example, by an external field generator arrangement, such as surface electrodes, by intra-body or intra-cardiac devices, such as an intracardiac catheter, or both. In these examples, the location sensing elements can constitute electrodes on the tracked devices that generate outputs received and processed by the mapping and navigation controller 90 to track the location of the various location sensing electrodes within the localization volume.

The EAM system 70 can be equipped for both magnetic and impedance tracking capabilities. In such examples, impedance tracking accuracy can, in some instances be enhanced by first creating a map of the electric field induced by the electric field generator within the cardiac chamber of interest using a probe equipped with a magnetic location sensor, as is possible using the RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

The electroporation catheter system 60 can be combined or integrated with the EAM system 70 to allow graphical representations of the electric fields that can be produced by the electroporation catheter 105 to be visualized on an electro-anatomical map of the patient's heart. The integrated system can include the capability to enhance the efficiency of clinical workflows, including enhancement of providing a visual representation to the clinician of ablation lesions of portions of the patient's heart created through irreversible electroporation. The integrated system can include generating the graphical representations of the electric fields that can be produced by the electroporation catheter 105, generating the anatomical maps including generating the electro-anatomical maps, and displaying information related to the location and electric field strengths of the electric fields that can be produced by the electroporation catheter 105.

The depiction of the electrophysiology system 50 shown in FIG. 1 is intended for illustration or a general overview of the various components of the system 50 and is not intended to imply that the disclosure is limited to any set of components or arrangement of the components. For example, additional hardware components, such as breakout boxes or workstations, can be included in the electrophysiology system 50.

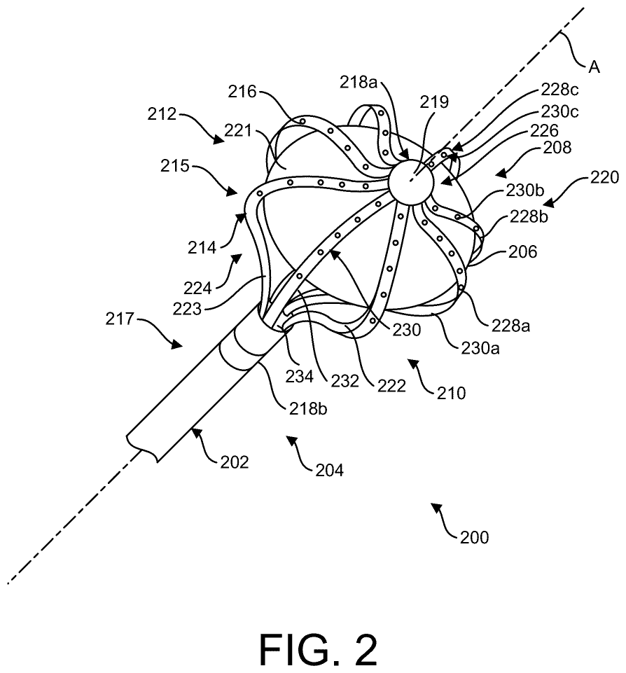

FIG. 2 illustrates an example electroporation catheter 200 for mapping and ablation of cardiac tissue, which can be an example of catheter 105 and used with the electrophysiology system 50. The example electroporation catheter 200 includes an elongated shaft 202 having a distal region 204. The elongate shaft 202 defines a longitudinal axis A. The distal region 204 is configured to be deployed proximate target tissue, such as within a chamber of the patient's heart or proximate the wall of a pulmonary vein ostium. An inflatable member 206 is operably coupled to the distal region 204. The inflatable member 206, such as a balloon, is transitionable between an inflated configuration 208 and an uninflated configuration. For example, the inflatable member 206 can be coupled to a source of fluid (e.g., saline) such as a reservoir and filled with the fluid in the inflated configuration 208 and void of the fluid in the uninflated configuration.

An electrode assembly 210 is operably coupled to the distal region 204. The electrode assembly 210 includes a plurality of electrodes 212 and a plurality of flexible support members 214. For instance, the electrode assembly 210 includes a sensing electrode assembly 215 including a plurality of sensing electrodes 216 of the plurality of electrodes 212 disposed on the plurality of flexible support members 214. The sensing electrode assembly 215 defines a distally located central hub portion 219 and the plurality of flexible support members 214 each including a distal end portion 221 extending from the central hub portion 219, and a proximal end portion 223 attached to and constrained by the shaft 204. Each of the plurality of flexible support members 214 includes the plurality of sensing electrodes 216. The electrode assembly 210 also includes an ablation electrode assembly 217 comprising a plurality of ablation electrodes 218 of the plurality of electrodes 212, the plurality of ablation electrodes 218 configured to generate an electric field to effect an electroporation ablation and operably coupled to the inflatable member 206. The flexible support members 214 are transitionable between an expanded configuration 220 and a collapsed configuration. For example, the flexible support members 214 can be configured in the collapsed configuration for travelling through the vasculature and configured in the expanded configuration when deployed proximate target tissue such as in a pulmonary vein ostium. The plurality of flexible support members 214 form an interior region, or cavity 222, such as in the expanded configuration 220, and the inflatable member 206 is disposed within the cavity 222.

The plurality of support members 214 can be formed as a basket 224 including the cavity 222 within the basket 224 in the expanded configuration 220. The inflatable member 206 is also disposed within the basket 224. The basket 224 can include a distal tip 226 and a proximal portion 228 operably coupled to the shaft 202. The distal tip 226 is configured to fit within a pulmonary vein ostium. The plurality of ablation electrodes 218 includes a first ablation electrode 218a disposed distal to the inflatable member 206 and a second ablation electrode 218b disposed proximal to the inflatable member 206. For example, the first ablation electrode 218a is disposed distal along the longitudinal axis A to the inflatable member 206 and the second ablation electrode 218b disposed proximal along the longitudinal axis A to the inflatable member 206. The electroporation catheter 200 can be configured to include one or more first, or distal ablation electrodes 218a distal to the inflatable member 206 and one or more second, or proximal ablation electrodes 218b disposed proximal to the inflation member 206. In the example, the first or distal ablation electrode 218a is disposed on the distal tip 226 of the basket 224, and the second or proximal ablation electrode 218b is coupled to the shaft 202 proximal to the basket 224. In one example, the first ablation electrode 218a is configured as one of a cathode and an anode and the second ablation electrode 218b is configured as the other of the cathode and the anode such that the electroporation catheter 200 is operable in a bipolar mode. In one example in the bipolar configuration, none of the first, or distal ablation electrodes 218a are proximal the inflatable member 206 and none of the second, or proximal ablation electrodes 218b are distal the inflatable member 206. In another example, the first and second ablation electrodes are configured as either the anode or as the cathode such that the electroporation catheter is operable in a monopolar mode, and an indifferent electrode is used to provide the other of the cathode and the anode. In one example, the plurality of flexible support members 214 are configured as a plurality of splines 228, and the splines 228 form the basket 224. Each spline 228a . . . 228n includes a corresponding set 230a . . . 230n of the plurality of sensing electrodes 216 longitudinally spaced along the spline 228a . . . 228n.

The splines 228 can be manufactured from a flat, flexible printed circuit with exposed, sensing electrodes 216 longitudinally spaced apart from each other at selected distances along the splines 228. In another embodiment, the splines 228 are configured from a longitudinal shape memory material such as nitinol with a circular cross section. The splines 228 are radially distributed in the electrode assembly 210. The number of splines and sensing electrodes can vary based on design. The flexible support members 214 can be deployed between the collapsed configuration and the expanded configuration using such mechanisms as a pull wire or rotatable member operable from the handle. A mid-longitudinal section of each spline 228 radially expands in the expanded configuration relative to the collapsed configuration in a known and repeatable three-dimensional shape for data collection. The greatest meridian of the basket 224 can be located at the longitudinal midpoint or at another location, such as towards the distal tip 226 relative the longitudinal midpoint. In one example, a set of sensing electrodes 230 are equidistantly spaced apart from each other. In another example, the set of sensing electrodes are disposed in a nonuniform distribution along the length of the spline, such as concentrated in a region likely to be proximate tissue when deployed or close to the endocardium. The sensing electrodes 216 are configured to detect relatively small voltages for a mapping process. The electrodes 212 can also include a locator electrode, such as a sensing electrode 216 configured to operate as a locator electrode, such as an electrode proximate the greatest meridian, on some splines 228. In one embodiment, the sensing electrodes are ring electrodes disposed around the splines, such as longitudinally extending splines with a circular cross section.

The shaft 202 can include a plurality of lead conductors that are electrically coupled to the electrodes 212. For example, the shaft 202 can include a plurality of lead conductors electrically coupled to the ablation electrodes 218, such as a first lead conductor to carry a first electrical signal associated with the first, or distal ablation electrode 218a, and a second lead conductor to carry a second electrical signal associated with the second, or proximal ablation electrode 218, the electrical signals to generate an electrical field with the ablation electrodes 218 for electroporation ablation as provided with the electroporation console 130. The shaft 202 also includes lead conductors electrically coupled to the sensing electrodes 216 to carry electrical cardiac signals obtained with the sensing electrodes 216 to the electroanatomical mapping system 70. Each sensing electrode 216 is electrically connected to cabling in the handle. Signals from each individual electrode can be independently available to the electroanatomical mapping system 70. This may be achieved by passing a lead conductor for each sensing electrode. Alternatively, the sensing electrodes can be multiplexed in the electrophysiological system 50 to minimize lead conductors included in the shaft 202.

The shaft 202 can also be configured to include a tube 232 that is in fluid communication with the inflatable member 206 via infusion port 234. The tube 232 can be in fluid communication with a reservoir containing a fluid. The inflatable member 206 in the uninflated configuration may be in a compact, deflated state suitable for advancement through vasculature. For example, the inflatable member 206 in the uninflated configuration may be substantially empty of fluid, such as sterile distilled or deionized water or saline. Fluid can enter the inflatable member 206 via the infusion port 234. The inflatable member 206 in the inflated configuration 208 holds a volume of the fluid that fills and inflates the inflatable member 206 to an appropriate size and shape such as having a diameter to contact walls of a pulmonary vein ostium under pressure from a syringe or other infusion device near the handle. The inflatable member 206 may transition to an intermediate configuration between the inflated and uninflated configuration, for example, to conform to a lumen or advance the device through vasculature. In some embodiments, the inflatable member 206 can be pressurized using a hand-operated syringe, pump, infusion device. In some examples, an infusion pressure can be between about 2 pounds per square inch (psi) and about 20 psi.

The electrodes 212 can be formed to include biocompatible metals such as titanium, palladium, gold, silver, platinum, or a platinum alloy. In some embodiments, the ablation electrodes 218 can have a biocompatible coating that permits capacitive voltage delivery with biphasic waveforms. Each electrode can include an electrical lead having sufficient electrical insulation to sustain an electrical potential difference of at least 700 V across its thickness without dielectric breakdown. In another example, the insulation on each of the electrical leads can sustain an electrical potential difference of between about 200 V to about 3,000 V across its thickness without dielectric breakdown. The body of the shaft 202 can be made of a flexible polymeric material such as those available under the trade designations of Teflon, Nylon, or PEBAX.

The inflatable member 206 can have an expandable structure and can be composed of any of a variety of insulating or dielectric materials including polyurethane or silicone, or can include other insulating or dielectric material including polyvinyl chloride (PVC), polyethylene (PE), cross-linked polyethylene, polyolefins, polyolefin copolymer (POC), polyethylene terephthalate (PET), polyester, nylon, polymer blends, polyester, polyimide, polyamides, polydimethylsiloxane (PDMS), or PEBAX. Together with the use of a nonconductive fluid such as distilled or deionized water to inflate the inflatable member 206, the inflatable member 206 in the inflated configuration 208 serves as an effective insulator during delivery of pulsed field ablation and drives the electric field to the region outside the inflatable member or balloon and surrounding the balloon such as towards the tissue rather than along an axis of the ablation electrode 218, longitudinal axis A, in the example, through the inflatable member 218.

According to various embodiments, the sensing and mapping aspects of the catheter 200 are similar or identifical to the corresponding features of the INTELLAMAP ORION™ mapping catheter marketed and sold by Boston Scientific Corporation. In other exemplary embodiments, the sensing and mapping aspects of the catheter 200 are similar or identical to the corresponding features of the Constellation™ Mapping Catheter marked and sold by Boston Scientific Corporation.

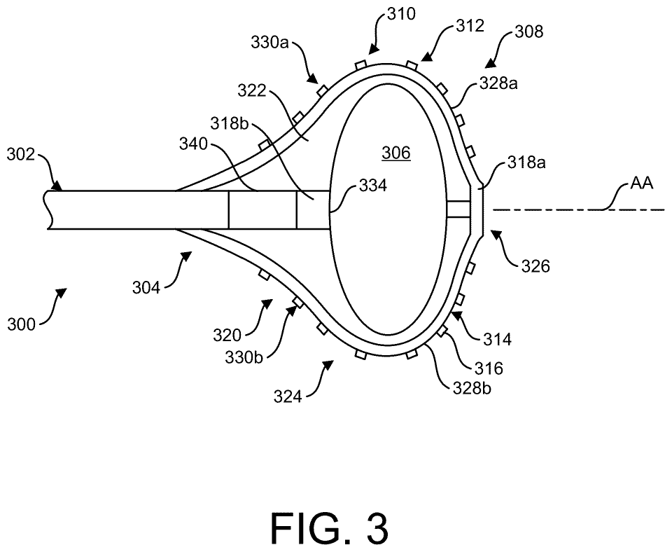

FIG. 3 illustrates another example electroporation catheter 300 for mapping and ablation of cardiac tissue, which can be an example of catheter 105 and used with the electrophysiology system 50. The example electroporation catheter 300 includes an elongated shaft 302 having a distal region 304. The elongate shaft 302 defines a longitudinal axis AA. The longitudinal axis AA is presented as a line passing through a centroid of a cross section of the shaft 302. The distal region 304 is configured to be deployed proximate target tissue, such as within a chamber of the patient's heart or proximate the wall of a pulmonary vein ostium. An inflatable member 306 is operably coupled to the distal region 304. The inflatable member 306, such as a balloon, is transitionable between an inflated configuration 308 and an uninflated configuration as the inflatable member 306 is selectively inflated or deflated with a fluid. An electrode assembly 310 is operably coupled to the distal region 304 configured in a sensing electrode assembly and an ablation electrode assembly. The electrode assembly 310 includes a plurality of electrodes 312 and a plurality of flexible support members 314. The sensing electrode assembly of electrode assembly 310 includes a sensing electrode assembly including a plurality of sensing electrodes 316 disposed on the plurality of flexible support members 314. The ablation electrode assembly of electrode assembly 310 includes a plurality of ablation electrodes 318 configured to generate an electric field to accomplish an electroporation ablation operably coupled to the inflation member 306. The flexible support members 314 are transitionable between an expanded configuration 320 and a collapsed configuration. The plurality of flexible support members 314 form an interior region or cavity 322, and the inflatable member 306 is disposed within the cavity.

The plurality of support members 314 can formed as a basket 324 including the cavity 322 within the basket 324 in the expanded configuration 320. In the example electroporation catheter 300, the shaft 302 extends into the cavity 322. The shaft 302 can include a shaft distal section 340 within the basket 324 that can include the infusion port 334 fluidically coupled to the inflatable member 306, and the inflatable member 306 is also disposed within the basket 324. The basket 324 can include a distal tip 326 and a proximal portion 328 operably coupled to the shaft 302. The distal tip 326 is configured to fit within a pulmonary vein ostium. The plurality of ablation electrodes 318 includes a first ablation electrode 318a disposed distal to the inflatable member 306, such as on the distal tip 326, and a second ablation electrode 318b disposed on the shaft distal section 304 within the basket 324 and the interior region 322. In one example, the first ablation electrode 318a is configured as one of a cathode and an anode and the second ablation electrode 318b is configured as the other of the cathode and the anode such that the electroporation catheter 300 is operable in a bipolar mode. In one example in the bipolar configuration, none of the first ablation electrodes 318a are proximal the inflatable member 306 and none of the second ablation electrodes 318b are distal the inflatable member 306. In one example, the plurality of flexible support members 314 are configured as a plurality of splines 328a, 328b, and the splines 328 form the basket 324. Each spline 328a, 238b includes a corresponding set 330a, 330b of the plurality of sensing electrodes 316 longitudinally spaced along the spline 328.

In one example, the shaft distal section 340 is fixed with respect to the shaft 302, and the proximal electrode 318b is not movable with respect to the basket 324. In another example, the shaft distal section 340 is movable along the longitudinal axis AA with respect to the basket 324, such as retractable and extendable within the cavity 322. For instance, shaft distal section 340 can be included as a coaxial element within the shaft 302 movable with respect to the shaft 302. The shaft 302 is fixedly coupled plurality of splines 328, and the shaft distal section 340 is axially movable with respect to the spines 328. In the example in which the distal ablation electrode 318a is coupled to the basket, the inflatable member 306 and proximal ablation electrode 318b are axially positionable with respect to the distal ablation electrode 318 to selectively configure an electric field to effect an electroporation ablation. The shape of the inflatable member 306 in the inflated configuration and the axial distance between the ablation electrodes 318 can be user selected during the electrophysiological procedure. In one example, the shaft distal section 340 can be moved with respect to the basket 324 via controls at the handle of the catheter 300.

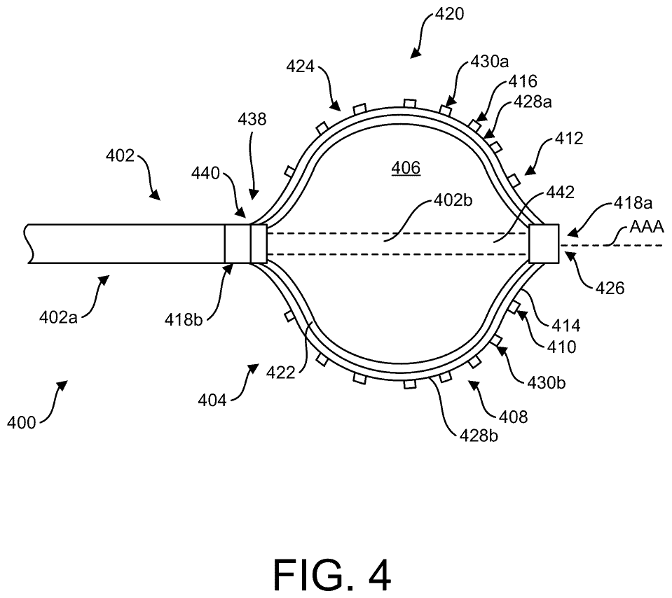

FIG. 4 illustrates another example electroporation catheter 400 for mapping and ablation of cardiac tissue, which can be an example of catheter 105 and used with the electrophysiology system 50. The example electroporation catheter 400 includes an elongated shaft 402 having a distal region 404. The elongate shaft 402 defines a longitudinal axis AAA. The longitudinal axis AAA is presented as a line passing through a centroid of a cross section of the shaft 402. The distal region 404 is configured to be deployed proximate target tissue, such as within a chamber of the patient's heart or proximate the wall of a pulmonary vein ostium. An inflatable member 406 is operably coupled to the distal region 404. The inflatable member 406, such as a balloon, is transitionable between an inflated configuration 408 and an uninflated configuration as the inflatable member 406 is selectively inflated or deflated with a fluid. An electrode assembly 410 is operably coupled to the distal region 404. The electrode assembly 410 includes a plurality of electrodes 412 configured as an sensing electrode assembly and an ablation electrode assembly. The sensing electrode assembly includes a plurality of sensing electrodes 416 disposed on a plurality of flexible support members 414. The ablation electrode assembly includes a plurality of ablation electrodes 418 of the plurality of electrodes 412 configured to generate an electric field to effect an electroporation ablation and operably coupled to the inflatable member 406. The flexible support members 414 are transitionable between an expanded configuration 420 and a collapsed configuration. The plurality of flexible support members 414 form a cavity 422, and the inflatable member 406 is disposed within the cavity.

The shaft 402 includes an outer shaft 402a defining a lumen, and an inner shaft 402b disposed within the lumen and coaxially with the outer shaft 402a. The inner shaft 402b is axially movable relative to the outer shaft 402a. The inflatable member 406 is coupled to outer shaft 402a at an outer shaft distal section 440. The lumen terminates at an infusion port 432 on the outer shaft distal section 440 within the chamber of the inflatable member 406. The inner shaft 402b is disposed within a chamber of the inflatable member 406, and the inflatable member is coupled to the inner shaft 402b at an inner shaft distal section 442. A first ablation electrode 418a is disposed on inner shaft 402b at the inner shaft distal section 442 distal to the inflatable member 406. The second ablation electrode is disposed on the outer shaft 402a at the outer shaft distal section 440 proximal to the inflatable member 406. The inner shaft 402b is movable axially with respect to the outer shaft 402a to selectively adjust the axial distance between the first ablation electrode 418a and the second ablation electrode 418b and the shape of the inflatable member 406 in the inflated configuration 408.

The plurality of support members 414 can be formed as a basket 424 including the cavity 422 within the basket 424. The inflatable member 406 is also disposed within the basket 424. The basket 424 can include a distal portion 426 coupled to the inner shaft 402b and a proximal portion 438 coupled to the outer shaft 402a. The distal portion 446 is configured to fit within a pulmonary vein ostium. In one example, the plurality of flexible support members 414 are configured as a plurality of splines 428, and the splines 428 form basket 424. Each spline 428a . . . 428n includes a corresponding set 430a . . . 430n of the plurality of sensing electrodes 416 longitudinally spaced along the spline 428a . . . 428n.

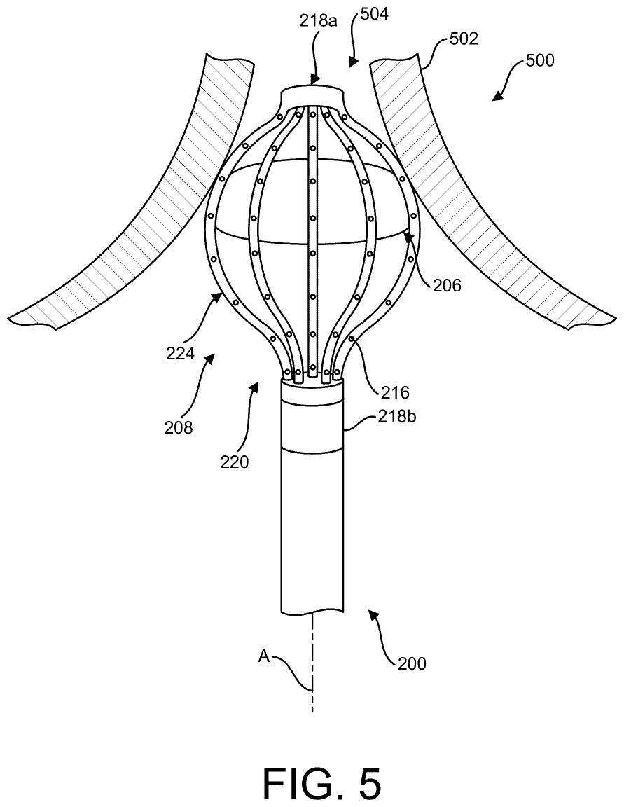

FIG. 5 illustrates catheter 200 in an expanded and inflated configuration deployed within cardiac tissue 500 such as a wall 502 defining a pulmonary vein ostium 504. The catheter 200 can include a plurality of combinations of configurations, such as uninflated and collapsed configurations, uninflated and expanded configurations, and inflated and expanded configurations. The catheter 200 may be in the uninflated and collapsed configurations as it travels through the vasculature. Once at the ostium, the catheter 200 can be deployed in the uninflated and expanded configurations, such as for mapping. The catheter 200 can be deployed with the inflatable member 206 and flexible support members 214 in the inflated and expanded configurations to configure the electric field to effect electroporation ablation. The catheter 200 can also be deployed with the inflatable member 206 and flexible support members 214 in the inflated and expanded configurations 208, 220 to substantially occlude the pulmonary vein ostium 504 and reduce a blood pool for mapping. In the example, the basket 224 is expanded and deployed against the pulmonary vein ostium wall 502 such that the first ablation electrode 218a is deployed within the pulmonary vein side of the ostium 504 and the second ablation electrode 218b is deployed on the left atrium side of the ostium 504. In this configuration, the longitudinal axis A is aligned with an axis of the pulmonary vein. The flexible support members 514 in the expanded configuration are configured to engage the pulmonary vein ostium wall 502. The inflatable member 206 is inflated with fluid to occlude, or substantially occlude, the ostium and, in some examples, contact the wall 502. In the case of a catheter with positionable ablation electrodes or a positionable inflatable member 206, the clinician is able to select a position of each to improve ablation or mapping efficiency. In the expanded and inflated configurations, 220, 208 as illustrated, a clinician can ablate tissue with the ablation electrodes 218 and obtain cardiac signals with the sensing electrodes 216 without maneuvering the catheter 200.

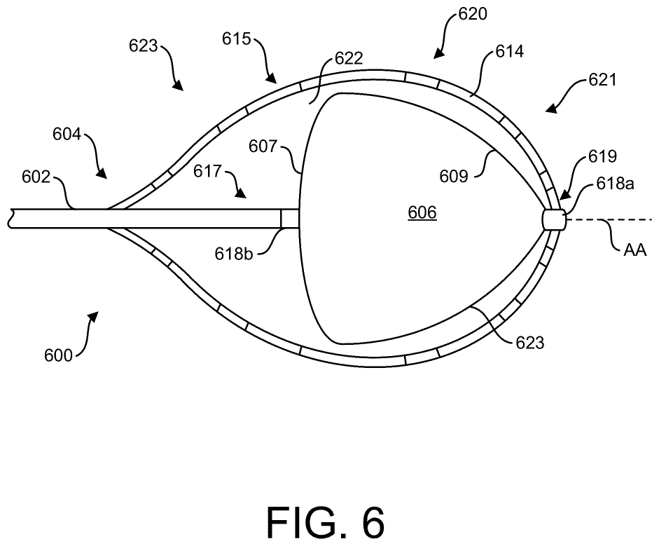

FIG. 6 illustrates another example electroporation catheter 600, which can be an example of catheter 300, for mapping and ablation of cardiac tissue, which can be an example of catheter 105 and used with the electrophysiology system 50. Catheter 600 can include features similar to catheter 300 of FIG. 3. For instance, catheter 600 includes an elongated shaft 602 having a distal region 604. A sensing electrode assembly 615 extends distally from the distal region 604 of the elongated shaft 602 and defines an interior region 622. The sensing electrode assembly 615 defines a distally located central hub portion 619 and a plurality of flexible support members 614. Each of the plurality of flexible support members 614 includes a distal end portion 621 that extends from the central hub portion 619 and a proximal end portion 623 attached to and constrained by the shaft 602. Also, each of the plurality of flexible support members 614 includes a plurality of sensing electrodes 616. In the illustrated example, the plurality of sensing electrodes 616 are ring electrodes that extend around the corresponding flexible support member 614. The flexible support members 614 are transitionable between an expanded configuration 620 and a collapsed configuration. The plurality of flexible support members 614 form a cavity or interior region 622 in the expanded configuration 620. An inflatable member 606 having a proximal portion 607 and a distal portion 609 is disposed with in the cavity 622. The inflatable member 606 transitionable between an inflated configuration 623 and an uninflated configuration. An ablation electrode assembly 617 is operably coupled to the inflatable member 606. The ablation electrode assembly 617 includes a proximal ablation electrode 618b disposed proximal to the inflatable member 606 and a distal ablation electrode 618a disposed distal to the inflatable member 606. In the example, the proximal ablation electrode 618b is disposed against the proximal portion 607 of the inflatable member 606 and a distal ablation electrode 618a is disposed against the distal portion 609 of the inflatable member 606. As illustrated in FIG. 6, the inflatable member is asymmetrical about a plane perpendicular to the axis AA and bisecting the distal portion 609 from the proximal portion 607. The ablation electrodes 618 are configured to generate an electric field to accomplish pulsed field ablation.

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An electroporation catheter for ablation of cardiac tissue, the electroporation catheter comprising:
an elongated shaft having a distal region;
a sensing electrode assembly extending distally from the distal region of the elongated shaft and defining an interior region, the sensing electrode assembly defining a distally located central hub portion and a plurality of flexible support members each including a distal end portion extending from the central hub portion, and a proximal end portion attached to and constrained by the shaft, each of the plurality of flexible support members including a plurality of sensing electrodes;
wherein the flexible support members are transitionable between an expanded configuration and a collapsed configuration, the plurality of flexible support members forming the interior region in the expanded configuration;
an inflatable member having a proximal portion and a distal portion, the inflatable member disposed within the interior region and operably coupled to the distal region, the inflatable member transitionable between an inflated configuration and an uninflated configuration; and
an ablation electrode assembly operably coupled to the inflatable member, the ablation electrode assembly having a proximal ablation electrode disposed proximal to the inflatable member and a distal ablation electrode disposed distal to the inflatable member, the ablation electrodes configured to generate an electric field to accomplish pulsed field ablation.

2. The electroporation catheter of claim 1, wherein the plurality of flexible support members form a basket having the cavity within the basket, and the inflatable member disposed within the basket.

3. The electroporation catheter of claim 2, wherein the basket includes a distal tip, and the first ablation electrode is disposed on the distal tip of the basket.

4. The electroporation catheter of claim 3, wherein the basket is operably coupled to the shaft, and the proximal ablation electrode is coupled to the shaft proximal to the basket.

5. The electroporation catheter of claim 1, wherein the distal ablation electrode is configured as one of a cathode and an anode and wherein the proximal ablation electrode is configured as the other of the cathode and the anode, and wherein the electroporation catheter is operable in a bipolar mode.

6. The electroporation catheter of claim 1, wherein the plurality of ablation electrodes includes one distal ablation electrode disposed distal to the inflatable member and one proximal ablation electrode disposed proximal to the inflatable member.

7. The electroporation catheter of claim 1, wherein the inflatable member is formed of an insulating material.

8. The electroporation catheter of claim 7, wherein the inflatable member in the inflated configuration includes a non-conductive fluid.

9. The electroporation catheter of claim 1, wherein the plurality of flexible support members includes a plurality of splines.

10. The electroporation catheter of claim 9, wherein each spline includes a set of the plurality of sensing electrodes longitudinally spaced along the spline.

11. An electrophysiology system, comprising:
an electroporation console configured to generate pulsed electrical signals for electroporation ablation;
an electroanatomical mapping system configured to receive cardiac signals; and
an electroporation catheter operably coupled to the electroporation console and the electroanatomical mapping system, the electroporation catheter comprising:
an elongated shaft having a distal region;
a sensing electrode assembly extending distally from the distal region of the elongated shaft and defining an interior region, the sensing electrode assembly defining a distally located central hub portion and a plurality of flexible support members each including a distal end portion extending from the central hub portion, and a proximal end portion attached to and constrained by the shaft, each of the plurality of flexible support members including a plurality of sensing electrodes;
wherein the flexible support members are transitionable between an expanded configuration and a collapsed configuration, the plurality of flexible support members forming the interior region in the expanded configuration;
an inflatable member having a proximal portion and a distal portion, the inflatable member disposed within the interior region and operably coupled to the distal region, the inflatable member transitionable between an inflated configuration and an uninflated configuration; and
an ablation electrode assembly operably coupled to the inflatable member, the ablation electrode assembly having a proximal ablation electrode disposed proximal to the inflatable member and a distal ablation electrode disposed distal to the inflatable member, the ablation electrodes configured to generate an electric field to accomplish pulsed field ablation.

12. The electrophysiological system of claim 11, and further comprising an infusion device, wherein the inflatable member is fluidically coupled to the infusion device to transition between the inflated configuration and the uninflated configuration in response to an infusion of fluid from the infusion device.

13. The electrophysiological system of claim 11, wherein an ablation electrode in the plurality of ablation electrodes is configured as a cathode and another ablation electrode in the plurality of ablation electrodes is configured as an anode, and the electroporation catheter is operable in a bipolar mode.

14. A method for an electrophysiological procedure, the method comprising:
providing an electroporation catheter comprising:
an elongated shaft having a distal region;
a sensing electrode assembly extending distally from the distal region of the elongated shaft and defining an interior region, the sensing electrode assembly defining a distally located central hub portion and a plurality of flexible support members each including a distal end portion extending from the central hub portion, and a proximal end portion attached to and constrained by the shaft, each of the plurality of flexible support members including a plurality of sensing electrodes;
wherein the flexible support members are transitionable between an expanded configuration and a collapsed configuration, the plurality of flexible support members forming the interior region in the expanded configuration;
an inflatable member having a proximal portion and a distal portion, the inflatable member disposed within the interior region and operably coupled to the distal region, the inflatable member transitionable between an inflated configuration and an uninflated configuration; and
an ablation electrode assembly operably coupled to the inflatable member, the ablation electrode assembly having a proximal ablation electrode disposed proximal to the inflatable member and a distal ablation electrode disposed distal to the inflatable member, the ablation electrodes configured to generate an electric field to accomplish pulsed field ablation;
deploying the electroporation catheter in the expanded configuration and the inflatable member in the inflated configuration into a pulmonary vein ostium;
delivering electroporation ablation energy to the ablation electrodes to ablate a wall of the pulmonary vein ostium with the deployed electroporation catheter in the expanded configuration and the inflatable member in the inflated configuration; and
obtaining cardiac signals with the sensing electrodes with the deployed electroporation catheter in the expanded configuration.

15. The method of claim 14, wherein deploying the electroporation catheter includes inflating the inflatable member into the cavity after expanding the flexible support members to form the cavity.

16. The method of claim 14, wherein deploying the electroporation catheter includes infusing the inflatable member with a fluid.

17. The method of claim 14, wherein the flexible support members include splines and deploying the electroporation catheter includes forming the basket in the expanded state.

18. The method of claim 14, wherein delivering electroporation ablation energy to the ablation electrodes to ablate a wall of the pulmonary vein ostium with the deployed electroporation catheter in the expanded configuration and the inflatable member in the inflated configuration includes occluding the pulmonary vein ostium.

19. The method of claim 14, wherein the inflatable member is formed of an insulating material, and wherein delivering electroporation ablation energy to the ablation electrodes to ablate a wall of the pulmonary vein ostium includes directing the electric field toward the wall of the pulmonary vein ostium with the inflatable member.

20. The method of claim 14, wherein the obtaining cardiac signals with the sensing electrodes includes occluding the pulmonary vein ostium.
